(19) United States
(12) Patent Application Publication
(10) Pub. No.: US 2026/0232214 A1
(43) Pub. Date: Aug. 13, 2026

(54) BIOMETRIC INFORMATION ACQUISITION APPARATUS AND NON-TRANSITORY COMPUTER-READABLE STORAGE MEDIUM STORING BIOMETRIC INFORMATION ACQUISITION PROGRAM

(71) Applicant: SEIKO EPSON CORPORATION, TOKYO (JP)

(72) Inventors: Akira IKEDA, Chino-shi (JP); Satoshi SHIMIZU, SHIOJIRI-SHI (JP)

(21) Appl. No.: 19/539,195

(22) Filed: Feb. 13, 2026

(30) Foreign Application Priority Data
Feb. 13, 2025 (JP) 2025-021362

(51) Int. Cl. A61B 5/024; G06T 7/246; G06T 7/73

(52) U.S. Cl. CPC A61B 5/02438; G06T 7/246; G06T 7/73

(57) ABSTRACT

A biometric information acquisition apparatus includes: a face recognition unit configured to identify a face image contained in image data and acquire three-dimensional coordinate data indicating positions of a plurality of feature points; and a detection unit configured to generate a plurality of pieces of feature point data in which the three-dimensional coordinate data is associated with a light amount value of a pixel contained in the image data, extract: measurement feature point data for detecting a pulse wave signal of a living body from the plurality of pieces of feature point data by using the three-dimensional coordinate data, and detect the pulse wave signal using the measurement feature point data.

The present application is based on, and claims priority from JP Application Serial Number 2025-021362, filed Feb. 13, 2025, the disclosure of which is hereby incorporated by reference herein in its entirety.

### BACKGROUND

### 1. Technical Field

The present disclosure relates to a biometric information acquisition apparatus and a non-transitory computer-readable storage medium storing a biometric information acquisition program.

### 2. Related Art

There is a pulse wave measurement apparatus that measures pulse waves of a living body. The pulse wave measurement apparatus is an example of a biometric information acquisition apparatus. A pulse wave measurement apparatus disclosed in JP-A-2021-183079 includes a reception unit, a display control unit, and a measurement unit. The reception unit receives an execution instruction of measurement of a pulse wave signal. The display control unit displays a captured image and a guide frame on a display unit. The measurement unit detects a skin region included in a face region contained in the guide frame. The measurement unit measures a pulse wave signal based on the skin region.

JP-A-2021-183079 is an example of the related art.

A face image region, which is an example of a face region, includes a part where a pulse wave signal is easily acquired and a part where a pulse wave signal is not easily acquired. Detection accuracy of the pulse wave measurement apparatus may be lowered depending on a location to be measured.

### SUMMARY

A biometric information acquisition apparatus according to the present disclosure includes: an imaging unit configured to image a living body and generate imaging data including a plurality of pieces of frame image data; a face recognition unit configured to identify a face image contained in the frame image data and acquire three-dimensional coordinate data indicating positions of a plurality of feature points contained in the face image; and a detection unit configured to generate a plurality of pieces of feature point data in which the three-dimensional coordinate data is associated with a light amount value of a pixel contained in the frame image data and detect a pulse wave signal of the living body based on the plurality of pieces of feature point data, in which the three-dimensional coordinate data includes a width component, a height component, and a depth component, and the detection unit extracts measurement feature point data from the plurality of pieces of feature point data by using the three-dimensional coordinate data contained in the feature point data and detects the pulse wave signal using the measurement feature point data.

A non-transitory computer-readable storage medium storing a biometric information acquisition program according to the present disclosure causes a computer, which is coupled to an imaging unit that images a living body and generates a plurality of frame image data, to execute the following steps of: image data; identifying a face image contained in the frame acquiring three-dimensional coordinate data indicating positions of a plurality of feature points contained in the face image; generating a plurality of pieces of feature point data in which the three-dimensional coordinate data is associated with a light amount value of a pixel contained in the frame image data; extracting measurement feature point data from the plurality of pieces of feature point data by using the three-dimensional coordinate data contained in the feature point data; and detecting a pulse wave signal using the measurement feature point data.

### BRIEF DESCRIPTION OF THE DRAWINGS

FIG. 1 is a view showing a schematic configuration of a measurement apparatus.

FIG. 2 is a block diagram showing the configuration of the measurement apparatus.

FIG. 3 is a view showing a captured image including face mesh information.

FIG. 4 is a view showing a part of the face mesh information.

FIG. 5 is a diagram showing an example of tone detection values.

FIG. 6 is a diagram showing an example of mesh point related information.

FIG. 7 is a diagram showing an example of a control flow executed by the measurement apparatus.

FIG. 8 is a diagram showing n-th face feature point time series data.

FIG. 9 is a diagram showing an example of an analysis procedure for detecting a pulse wave signal.

FIG. 10 is a diagram showing an analysis result of a noise-removed signal.

FIG. 11 is a diagram showing an example of a control flow executed by the measurement apparatus.

FIG. 12 is a diagram showing an example of a control flow executed by the measurement apparatus.

FIG. 13 is a diagram showing an example of a control flow executed by the measurement apparatus.

FIG. 14 is a diagram showing an example of a control flow executed by the measurement apparatus.

FIG. 15 is a diagram showing an example of a control flow executed by the measurement apparatus.

FIG. 16 is a diagram showing an example of a control flow executed by the measurement apparatus.

### DESCRIPTION OF EMBODIMENTS

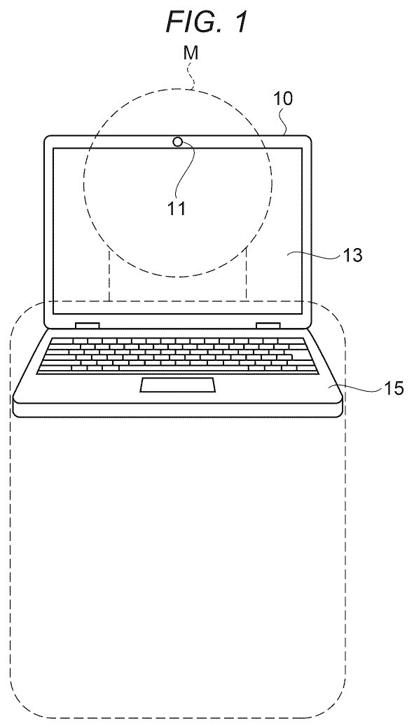

FIG. 1 shows a schematic configuration of a measurement apparatus 10. The measurement apparatus 10 corresponds to an example of a biometric information acquisition apparatus. The measurement apparatus 10 detects a pulse wave signal from a measurement operator M by using moving image data. The pulse wave signal is a signal indicating pulse waves of the measurement operator M. The measurement operator M corresponds to an example of a living body. The measurement apparatus 10 calculates biometric information of the measurement operator M based on a pulse wave signal. The biometric information includes pulse, pulse fluctuation, oxygen saturation concentration, blood pressure, and the like. The measurement apparatus 10 may evaluate sleep apnea syndrome or the like based on the biometric information. The measurement apparatus 10 displays the biometric information calculated based on the pulse wave signal.

The measurement apparatus 10 is implemented by an information processing apparatus such as a personal computer. The measurement apparatus 10 shown in FIG. 1 is a laptop computer, but is not limited thereto. The measurement apparatus 10 may be an apparatus having a function of capturing a moving image or an apparatus that can be coupled to a device for capturing a moving image. The measurement apparatus 10 is implemented by a desktop personal computer, a tablet terminal, a smartphone, or the like. The measurement apparatus 10 includes an imaging unit 11, a display unit 13, and an input unit 15. The measurement apparatus 10 may include a communication unit (not shown), or the like.

The imaging unit 11 images the measurement operator M by receiving reflected light, external light, or the like reflected by the measurement operator M or the like as detection light. The imaging unit 11 generates moving image data including the face of the measurement operator M. The moving image data is data for displaying a moving image, and includes a plurality of pieces of image data. The moving image data corresponds to an example of imaging data. The moving image data is implemented by multiple sets of image data. The imaging unit 11 generates the moving image data including a plurality of pieces of image data. The image data corresponds to an example of frame image data. The image data includes a plurality of output values that are output in units of pixels. The image data is data used to cause the display unit 13 to display a captured image 100. The captured image 100 is a still image. The imaging unit 11 captures images at a predetermined frame rate to generate the moving image data. The imaging unit 11 corresponds to an example of an imager.

The imaging unit 11 is, for example, a camera including an optical element, an imaging element, and the like. The optical element condenses light on the imaging element. The imaging element converts detection light into an output value of an electric signal. The imaging element generates an output value for each of a plurality of pixels. An output value of each pixel indicates a light intensity of each pixel. The imaging element generates an output value of each pixel. The imaging element includes a charge coupled device (CCD), a complementary metal oxide semiconductor (CMOS), and the like. The output value includes tone values of a plurality of color light beams. The imaging unit 11 generates tone values of a plurality of color light beams for each pixel. The plurality of color light beams are, for example, red light, green light, and blue light. The red light, the green light, and the blue light have different wavelength bands. The red wavelength band, which is a wavelength band of the red light, is 600 nm to 800 nm. The green wavelength band, which is a wavelength band of the green light, is 520 nm to 550 nm. The blue wavelength band, which is a wavelength band of the blue light, is 430 nm to 490 nm. The imaging unit 11 may include infrared (IR) light or the like. The imaging unit 11 generates a red tone value that is a tone value of the red light, a green tone value that is a tone value of the green light, and a blue tone value that is a tone value of the blue light for each pixel. The image data includes an output value for each pixel including the red tone value, the green tone value, and the blue tone value. The image data includes luminance of each pixel.

The imaging unit 11 shown in FIG. 1 is a camera incorporated in the measurement apparatus 10, but is not limited thereto. The imaging unit 11 may be an external camera coupled to the measurement apparatus 10. The external camera is a near-infrared camera, a web camera, a smartphone camera, or the like.

The display unit 13 displays various kinds of information such as the captured image 100. The display unit 13 displays various kinds of biometric information based on a pulse wave signal. The display unit 13 may display a comment or the like based on the biometric information. The display unit 13 is implemented by a liquid crystal panel, an organic electro-luminescence (EL) panel, or the like. The display unit 13 may have a touch input function. When the touch input function is provided, the display unit 13 functions as the input unit 15. The display unit 13 shown in FIG. 1 is provided in the measurement apparatus 10, but is not limited thereto. The display unit 13 may be a display externally attached to the measurement apparatus 10.

The input unit 15 receives various input operations performed by the measurement operator M. The input unit 15 generates various input signals according to the input operations. The input unit 15 shown in FIG. 1 is a keyboard provided in the measurement apparatus 10, but is not limited thereto. The input unit 15 may be a mouse, a keyboard, a touch panel, a pen tablet, or the like coupled to the measurement apparatus 10.

The measurement operator M operates the measurement apparatus 10 at a position where the measurement operator faces the imaging unit 11 of the measurement apparatus 10. The measurement operator M operates the measurement apparatus 10 when the measurement apparatus 10 is caused to detect biometric information. The measurement operator M may operate the measurement apparatus 10 when performing a task such as document creation. The measurement apparatus 10 detects the biometric information related to the measurement operator M in the background when the measurement operator M is performing a task such as document creation. The measurement apparatus 10 can detect the biometric information related to the measurement operator M in a typical active state by detecting the biometric information in the background.

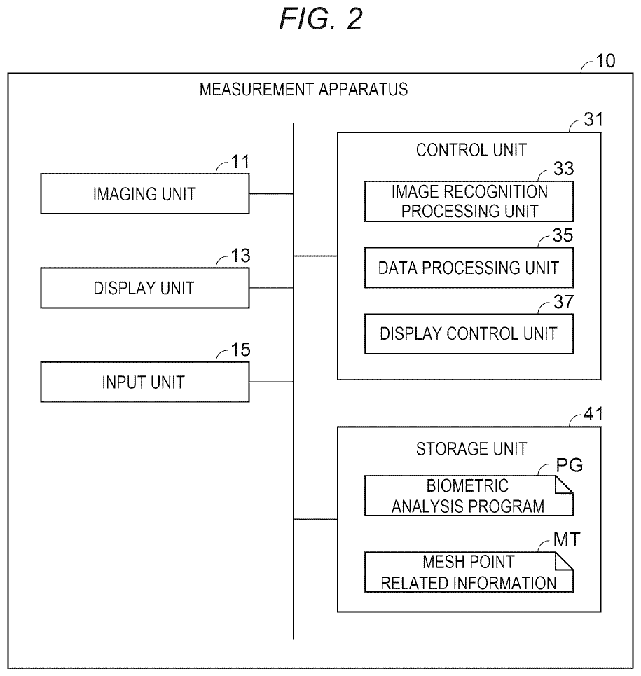

FIG. 2 is a block diagram showing the configuration of the measurement apparatus 10. The measurement apparatus 10 includes the imaging unit 11, the display unit 13, the input unit 15, a control unit 31, and a storage unit 41.

The imaging unit 11 transmits moving image data to the control unit 31. The imaging unit 11 transmits the moving image data to the control unit 31 at a predetermined timing. The imaging unit 11 may transmit image data contained in the moving image data to the control unit 31 at predetermined time intervals. The imaging unit 11 transmits image data including a red tone value, a green tone value, and a blue tone value for each pixel to the control unit 31. The imaging unit 11 may transmit the moving image data to the storage unit 41 and store the moving image data in the storage unit 41.

The display unit 13 displays various images under the control of the control unit 31. The display unit 13 receives display data from the control unit 31 and displays various images based on the display data. The display unit 13 may display a moving image captured by the imaging unit 11 based on the moving image data. The display unit 13 may display the captured image 100 based on image data contained in the moving image data.

The input unit 15 transmits an input signal to the control unit 31. The input unit 15 transmits the input signal to the control unit 31 to cause the control unit 31 to perform various types of control. For example, the input unit 15 transmits a display instruction signal for displaying biometric information to the control unit 31. The display instruction signal is an example of an input signal. Based on the display instruction signal, the control unit 31 generates biometric information display data for causing the display unit 13 to display biometric information based on a pulse wave signal. The control unit 31 transmits the biometric information display data to the display unit 13. The display unit 13 displays a screen including the biometric information based on the biometric information display data.

The control unit 31 is a controller that controls an operation of each unit. The control unit 31 is, for example, a processor including a central processing unit (CPU). The control unit 31 is implemented by one or more processors. The control unit 31 is communicatively coupled to the imaging unit 11, the display unit 13, and the like. The control unit 31 functions as an image recognition processing unit 33, a data processing unit 35, and a display control unit 37 by executing a biometric analysis program PG. The control unit 31 may function as a functional unit other than the image recognition processing unit 33, the data processing unit 35, and the display control unit 37 by executing the biometric analysis program PG. The control unit 31 corresponds to an example of a computer.

The image recognition processing unit 33 acquires the moving image data transmitted from the imaging unit 11. The image recognition processing unit 33 acquires a plurality of pieces of image data contained in the moving image data. The image recognition processing unit 33 identifies a face image contained in the image data. The image recognition processing unit 33 identifies a face image by executing face recognition processing on the image data. The face recognition processing is a processing of extracting a face image region 121 by detecting a face image feature point contained in the image data and matching the face image feature point with a face image database registered in advance. The face image database is a database that stores information related to a face image feature point used for face recognition. The face image feature point contained in the image data is, for example, a position and a contour of eye, nose, and mouth. The face image database is stored in the storage unit 41 in advance. When the measurement apparatus 10 is coupled to a server via a network, the face image database may be stored in the server in advance. The face image region 121 is a region where the face of the measurement operator M is displayed. The image recognition processing unit 33 identifies the face image region 121 by executing face recognition processing. The image recognition processing unit 33 corresponds to an example of a face recognition unit.

In the face recognition processing, for example, Face Mesh included in Media Pipe provided by Google (hereinafter, referred to as Face Mesh) is used. Face Mesh is a machine learning model that detects a key point of a face from an image. The image recognition processing unit 33 executes the face recognition processing using Face Mesh on each of a plurality of pieces of image data, and acquires the face image region 121 and face mesh information 131. The face mesh information 131 is represented by a plurality of key points contained in the face image region 121.

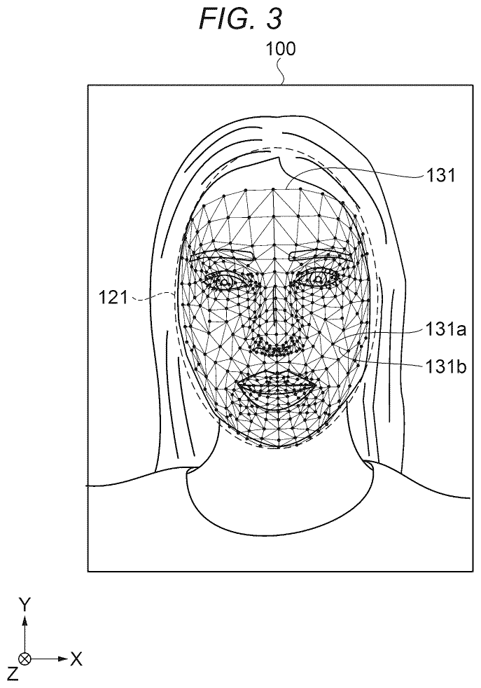

FIG. 3 shows the captured image 100 including the face mesh information 131. FIG. 3 shows an example of the face mesh information 131. The face mesh information 131 varies depending on the measurement operator M. FIG. 3 shows mesh points 131a and mesh lines 131b indicating the face mesh information 131.

FIG. 3 shows an XYZ coordinate system. An X axis is an axis along a width direction of the captured image 100. A Y axis is an axis along a height direction of the captured image 100. A Z axis is an axis along a vertical axis passing through the center of an imaging element in the imaging unit 11.

The mesh point 131a is a point corresponding to a key point. The number of key points is, for example, 468. The image recognition processing unit 33 specifies a plurality of the mesh points 131a contained in the face image region 121. The image recognition processing unit 33 specifies a plurality of the mesh points 131a in each of a plurality of pieces of image data. The number of mesh points 131a specified in each of the plurality of pieces of image data is the same. A predetermined code is assigned to each of the plurality of mesh points 131a. The plurality of mesh points 131a to which the same code is assigned correspond to the same position in the face image region 121. The mesh point 131a corresponds to an example of a feature point. The mesh line 131b is a line connecting two adjacent mesh points 131a.

The mesh point 131a is represented by three-dimensional coordinates including a width component, a height component, and a depth component with one mesh point 131a among the plurality of mesh points 131a as an origin. FIG. 3 shows the mesh points 131a of three-dimensional coordinates projected onto a two-dimensional plane. Each of the plurality of mesh points 131a is represented by mesh point coordinates including a width component, a height component, and a depth component. The mesh point coordinates correspond to an example of three-dimensional coordinate data.

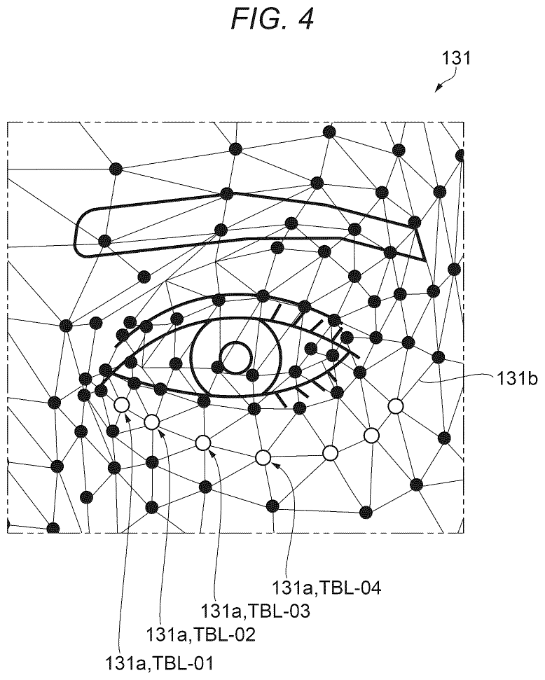

FIG. 4 shows a part of the face mesh information 131. FIG. 4 shows the face mesh information 131 near the left eye of the measurement operator M in an enlarged manner. FIG. 4 shows the plurality of mesh points 131a and a plurality of the mesh lines 131b. FIG. 4 shows code names of some mesh points 131a among the plurality of mesh points 131a.

FIG. 4 shows code names assigned to some mesh points 131a. The code names assigned to the mesh points 131a are, for example, a first code name TBL-01, a second code name TBL-02, a third code name TBL-03, and a fourth code name TBL-04. The code names shown in FIG. 4 indicate an eye bag of the left eye of the measurement operator M. A configuration of the code name can be set as appropriate. The image recognition processing unit 33 assigns a unique code name to each of the plurality of mesh points 131a.

The image recognition processing unit 33 shown in FIG. 2 calculates mesh point coordinates indicating a position of each of the plurality of mesh points 131a. The image recognition processing unit 33 calculates relative mesh point coordinates with a freely set point among the plurality of mesh points 131a as an origin. The image recognition processing unit 33 converts the relative mesh point coordinates into mesh point coordinates on the image data. A method of calculating the mesh point coordinates will be described later.

The data processing unit 35 shown in FIG. 2 acquires the moving image data transmitted from the imaging unit 11. The data processing unit 35 acquires a plurality of pieces of image data contained in the moving image data. The data processing unit 35 acquires the red tone value, the green tone value, the blue tone value, luminance of a pixel, and the like for each pixel contained in the image data. The data processing unit 35 appropriately executes data processing such as correction processing on the red tone value, the green tone value, the blue tone value, and the like.

The data processing unit 35 detects a pulse wave signal of the measurement operator M. The data processing unit 35 analyzes biometric information based on the pulse wave signal. The data processing unit 35 corresponds to an example of a detection unit. The data processing unit 35 acquires, from the imaging unit 11, image data implemented by output values for each pixel including a red tone value, a green tone value, and a blue tone value. The data processing unit 35 acquires the face mesh information 131 from the image recognition processing unit 33. The data processing unit 35 associates the mesh points 131a contained in the face mesh information 131 with the output values of pixels. The output value corresponds to an example of a light amount value. The data processing unit 35 associates the mesh points 131a with the output values of the pixels for each of the plurality of pieces of image data.

The data processing unit 35 tracks the predetermined mesh point 131a contained in the face mesh information 131 for each of the plurality of pieces of image data. The plurality of pieces of image data are generated in time series. The data processing unit 35 tracks a position of the predetermined mesh point 131a contained in each of the plurality of pieces of image data generated in time series by specifying the predetermined mesh point 131a in each of the plurality of pieces of image data.

The data processing unit 35 may track one mesh point 131a among the plurality of mesh points 131a, or may track all of the plurality of mesh points 131a. The data processing unit 35 may track one or more mesh points 131a selected in advance based on a predetermined condition among the plurality of mesh points 131a. The selection of the mesh point 131a is appropriately set.

The data processing unit 35 associates the predetermined mesh point 131a with an output value of a pixel for each piece of image data. The data processing unit 35 acquires an output value associated with the predetermined mesh point 131a. The data processing unit 35 acquires an output value associated with the predetermined mesh point 131a for each piece of image data. The data processing unit 35 acquires a time series output value associated with the predetermined mesh point 131a as face feature point time series data by acquiring an output value associated with the predetermined mesh point 131a for each piece of image data generated in time series.

The data processing unit 35 detects a pulse wave signal of the measurement operator M using the face feature point time series data associated with the predetermined mesh point 131a. The data processing unit 35 generates a detection value using a plurality of output values contained in the face feature point time series data. The detection value includes a calculated tone detection value. The detection value may be face feature point time series data associated with one mesh point 131a, or may be a value calculated based on a plurality of pieces of face feature point time series data respectively associated with the plurality of mesh points 131a. The calculated value is, for example, an average value of the output values associated with the plurality of mesh points 131a for each piece of image data. The detection value is calculated for each piece of image data contained in the moving image data. The data processing unit 35 acquires a pulse wave signal based on the detection value of the predetermined mesh point 131a for each of the plurality of pieces of image data.

The data processing unit 35 detects the pulse wave signal using each tone detection value contained in the detection values. For example, the data processing unit 35 detects the pulse wave signal by using at least one of a red tone detection value Dr, a green tone detection value Dg, and a blue tone detection value Db. The red tone detection value Dr is calculated using the red tone value. The green tone detection value Dg is calculated using the green tone value. The blue tone detection value Db is calculated using the blue tone value. For example, the pulse wave signal is detected based on the green tone detection value Dg. The pulse wave signal may be detected based on a difference between the green tone detection value Dg and at least one of the red tone detection value Dr and the blue tone detection value Db.

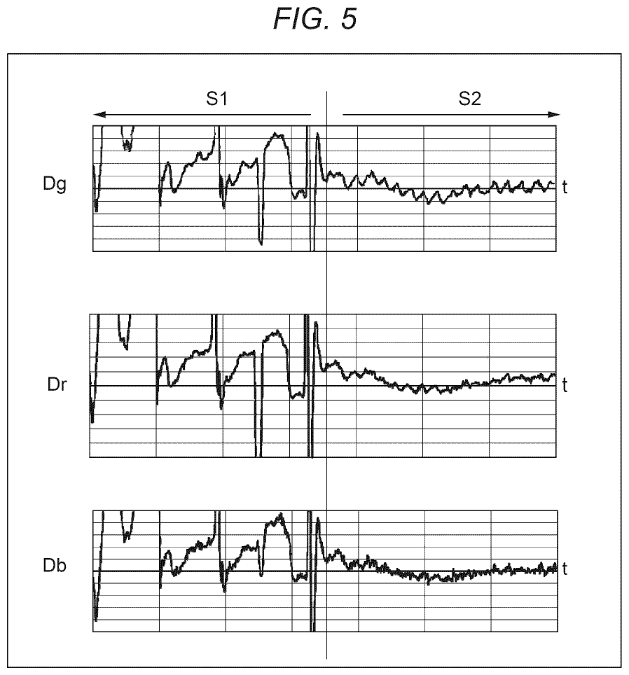

FIG. 5 shows an example of tone detection values. FIG. 5 shows the red tone detection value Dr, the green tone detection value Dg, and the blue tone detection value Db. FIG. 5 shows over-time changes in the red tone detection value Dr, the green tone detection value Dg, and the blue tone detection value Db in the form of waveform signals. FIG. 5 shows the red tone detection value Dr, the green tone detection value Dg, and the blue tone detection value Db in a body motion section S1, and the red tone detection value Dr, the green tone detection value Dg, and the blue tone detection value Db in a rest section S2. The body motion section S1 is a section in which a face moves or a facial expression changes. The rest section S2 is a section in which a face motion or a facial expression change is smaller than a predetermined change amount.

FIG. 5 shows the green tone detection value Dg. The green tone detection value Dg corresponds to a light amount value of the green light contained in the output value at one or more predetermined mesh points 131a. The green tone detection value Dg is contained in a detection value.

As shown in FIG. 5, in the body motion section S1, the green tone detection value Dg varies due to an influence of a body motion. A pulse wave signal contained in the green tone detection value Dg is less likely to be detected due to variation noises. In the rest section S2, the influence of variation noises on the green tone detection value Dg due to a body motion is reduced, and a pulse wave signal can be detected.

FIG. 5 shows the red tone detection value Dr. The red tone detection value Dr corresponds to a light amount value of the red light contained in the output value at one or more predetermined mesh points 131a. The red tone detection value Dr is contained in a detection value.

As shown in FIG. 5, in the body motion section S1, the red tone detection value Dr varies due to an influence of a body motion. A pulse wave signal contained in the red tone detection value Dr is less likely to be detected due to variation noises. In the rest section S2, the influence of the variation noises on the red tone detection value Dr due to a body motion is reduced, but an SN ratio is small, and thus it is less likely to detect a pulse wave signal.

FIG. 5 shows the blue tone detection value Db. The blue tone detection value Db corresponds to a light amount value of the blue light contained in the output value at one or more predetermined mesh points 131a. The blue tone detection value Db is contained in a detection value.

As shown in FIG. 5, in the body motion section S1, the blue tone detection value Db varies due to an influence of a body motion. A pulse wave signal contained in the blue tone detection value Db is less likely to be detected due to variation noises. In the rest section S2, the influence of the variation noises on the blue tone detection value Db due to a body motion is reduced, but an SN ratio is small, and thus it is less likely to detect a pulse wave signal.

The data processing unit 35 detects a pulse wave signal by using the red tone detection value Dr, the green tone detection value Dg, and the blue tone detection value Db shown in FIG. 5. The data processing unit 35 detects the pulse wave signal in an analysis procedure to be described later.

The data processing unit 35 calculates biometric information such as a pulse by calculating a cycle, an amplitude, and the like of the pulse wave signal. The data processing unit 35 transmits the biometric information including the pulse wave signal to the display control unit 37. The data processing unit 35 may store the biometric information and the like in the storage unit 41.

The display control unit 37 shown in FIG. 2 controls a display operation performed by the display unit 13. The display control unit 37 acquires the biometric information including the pulse wave signal from the data processing unit 35. The display control unit 37 generates biometric information display data including the biometric information. The display control unit 37 transmits the biometric information display data to the display unit 13. The display control unit 37 causes the display unit 13 to display the biometric information display data. The display control unit 37 can notify the measurement operator M of a detection result of the biometric information by causing the display unit 13 to display the biometric information display data.

The display control unit 37 may generate message data indicating an operation state of the biometric analysis program PG. The message data includes a start message, an execution message, an end message, and the like. The start message indicates that the detection of the biometric information is started. The execution message indicates that the biometric information is being detected. The end message indicates that the detection of the biometric information is ended. The display control unit 37 transmits the message data to the display unit 13. The display control unit 37 causes the display unit 13 to display the message data.

The storage unit 41 stores various programs, various kinds of data, and the like. The storage unit 41 corresponds to an example of a storage. The storage unit 41 stores the biometric analysis program PG and mesh point related information MT. The storage unit 41 stores a document creation program, a spreadsheet program, and the like. The storage unit 41 may store various kinds of data such as moving image data and biometric information. The storage unit 41 may store a face image database. The storage unit 41 is implemented by a semiconductor memory such as a random access memory (RAM) and a read only memory (ROM). The storage unit 41 may include a hard disk drive (HDD). The storage unit 41 may function as a work area for the control unit 31.

The biometric analysis program PG is a program for causing the measurement apparatus 10 to detect a pulse wave signal. The biometric analysis program PG is executed by the control unit 31. When the biometric analysis program PG is executed by the control unit 31, the control unit 31 functions as various functional units. The biometric analysis program PG is used to detect various kinds of biometric information based on the pulse wave signal. The biometric analysis program PG may be executed in the background when the control unit 31 executes a document creation program or the like. The biometric analysis program PG corresponds to an example of a biometric information acquisition program.

The mesh point related information MT is information related to the adjacent mesh points 131a among the plurality of mesh points 131a. The mesh point related information MT is generated when the image recognition processing unit 33 executes face recognition processing, and is stored in the storage unit 41. The mesh point related information MT is used when the data processing unit 35 selects one or more of the mesh points 131a from the plurality of mesh points 131a.

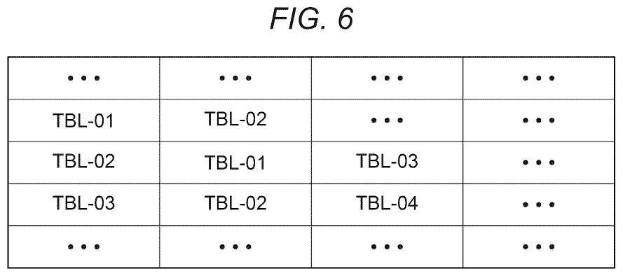

FIG. 6 shows an example of the mesh point related information MT. FIG. 6 shows the mesh point related information MT in a table format. FIG. 6 shows information related to the mesh points 131a of the first code name TBL-01, the second code name TBL-02, the third code name TBL-03, and the fourth code name TBL-04 shown in FIG. 4.

The mesh point related information MT shows the mesh point 131a and the mesh points 131a adjacent thereto. Two adjacent mesh points 131a are coupled by the mesh line 131b. The mesh point related information MT indicates two or three or more mesh points 131a coupled by the mesh line 131b. For example, the mesh point related information MT indicates that the mesh point 131a of the first code name TBL-01 is located at a position adjacent to the mesh point 131a of the second code name TBL-02. The mesh point related information MT indicates that the mesh point 131a of the second code name TBL-02 is located at a position adjacent to the mesh point 131a of the first code name TBL-01 and the mesh point 131a of the third code name TBL-03.

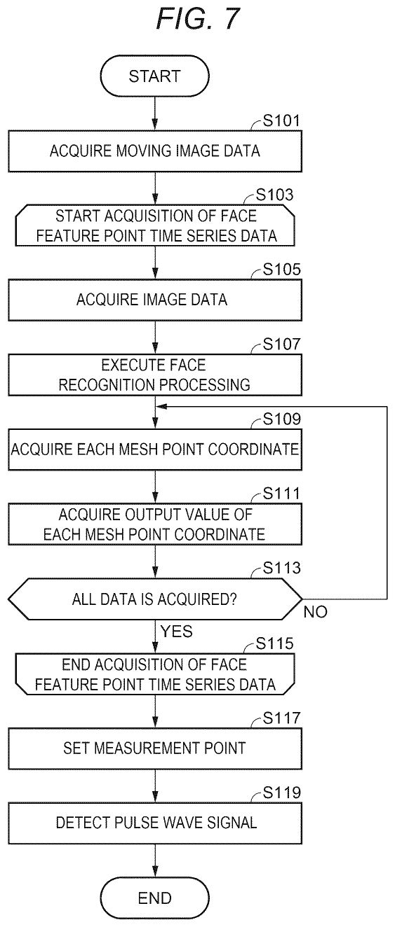

FIG. 7 shows an example of a control flow executed by the measurement apparatus 10. FIG. 7 shows a control flow for acquiring a pulse wave signal using an output value of a pixel corresponding to the mesh point 131a. The control flow is executed by executing the biometric analysis program PG. FIG. 7 shows the control flow in a flowchart.

In step S101, the measurement apparatus 10 acquires moving image data. The measurement apparatus 10 causes the imaging unit 11 to generate the moving image data. The imaging unit 11 images the measurement operator M and generates the moving image data. The moving image data contains a plurality of pieces of image data. The plurality of pieces of image data are generated in time series. The imaging unit 11 generates the plurality of pieces of image data. When a frame rate at which the imaging unit 11 generates the moving image data is, for example, 30 frames per second (fps) and a measurement time is 8 seconds, the number of pieces of image data is 240. The imaging unit 11 transmits the moving image data to the control unit 31.

After acquiring the moving image data, the measurement apparatus 10 starts acquiring the face feature point time series data in step S103. The data processing unit 35 of the control unit 31 acquires the moving image data transmitted from the imaging unit 11. The control unit 31 acquires the plurality of pieces of image data contained in the moving image data. The data processing unit 35 acquires the face feature point time series data of each mesh point 131a by executing the processing from step S103 to step S111. Details of the face feature point time series data will be described later.

In step S105, the measurement apparatus 10 acquires the image data. The image recognition processing unit 33 of the control unit 31 sequentially acquires the image data generated in time series. When the moving image data includes k pieces of image data, the image recognition processing unit 33 sequentially acquires the first image data to the k-th image data. k is a freely set integer. k is set according to a frame rate of the moving image data and a measurement time.

After acquiring the image data, the measurement apparatus 10 executes the face recognition processing in step S107. The image recognition processing unit 33 executes the face recognition processing on each of the plurality of pieces of image data. The image recognition processing unit 33 generates the face mesh information 131 contained in each piece of image data. The face mesh information 131 includes the plurality of the mesh points 131a and the plurality of the mesh lines 131b. When the face image region 121 is contained in each piece of image data, the number of the plurality of mesh points 131a contained in each piece of image data is the same.

After executing the face recognition processing, the measurement apparatus 10 acquires mesh point coordinates of each mesh point 131a in step S109. The image recognition processing unit 33 acquires the face mesh information 131 of each piece of image data. The image recognition processing unit 33 acquires the mesh point coordinates of the plurality of mesh points 131a contained in each piece of image data. The image recognition processing unit 33 generates relative mesh point coordinates with the predetermined mesh point 131a in the face mesh information 131 as an origin. The face mesh information 131 includes N mesh points 131a including the n-th mesh point 131a. n-th relative mesh point coordinates Crn, which are relative mesh point coordinates of the n-th mesh point 131a contained in one piece of image data, are represented by the following formula (1).

$$Crn = ( An , Bn , Wn ) \qquad ( 1 )$$

Here, A is a distance in a width direction from the mesh point 131a set as the origin, and is an example of a width component. B is a distance in a height direction from the mesh point 131a set as the origin, and is an example of a height component. W is a distance in a depth direction from the mesh point 131a set as the origin. n is any integer from 1 to N. Here, N is an integer of 2 or more.

After acquiring the n-th relative mesh point coordinates Crn, the image recognition processing unit 33 converts the n-th relative mesh point coordinates Crn into mesh point coordinates on image data with a freely set position in the image data as an origin. n-th mesh point coordinates Cn, which are mesh point coordinates on the image data, are represented by the following formula (2).

$$Cn = ( xn , yn , zn ) \qquad ( 2 )$$

Here, x is a distance along an X axis from the origin in the image data. xn is a distance along the X axis from the origin in the image data of the mesh point 131a of the n-th mesh point coordinates Cn. x and xn are examples of a width component. y is a distance along a Y axis from the origin in the image data. yn is a distance along the Y axis from the origin in the image data of the mesh point 131a of the n-th mesh point coordinates Cn. y and yn are examples of a height component. z is a value calculated based on W. For example, z is a difference value with an average value obtained by averaging W of the plurality of mesh points 131a as the origin. z is calculated by converting the difference value into the same unit as x and y. zn is a difference value from the average value to the mesh point 131a of the n-th mesh point coordinates Cn. z and zn are examples of a depth component.

The n-th mesh point 131a contained in each of the plurality of pieces of image data indicates the same position in the face image region 121. The n-th mesh point coordinates Cn of each of the plurality of pieces of image data vary depending on a position and an n orientation of the face of the measurement operator M in the image data. The control unit 31 can track the n-th mesh point 131a in time series by acquiring the n-th mesh point coordinates Cn contained in each piece of image data. The control unit 31 can track positions of all the mesh points 131a in time series by acquiring the n-th mesh point coordinates Cn of each of the plurality of pieces of image data.

After acquiring the mesh point coordinates, the measurement apparatus 10 acquires output values of the mesh point coordinates in step S111. The data processing unit 35 acquires the mesh point coordinates from the image recognition processing unit 33. The data processing unit 35 acquires an output value of a pixel corresponding to the mesh point coordinates. The data processing unit 35 may acquire output values of a pixel corresponding to the mesh point coordinates and peripheral pixels that are pixels in a predetermined region with respect to the pixel corresponding to the mesh point coordinates. The predetermined region is set in advance. When the output values of the peripheral pixels are acquired, for example, the data processing unit 35 acquires, as an output value, an average value of the output values of the pixels corresponding to the mesh point coordinates and the output values of the peripheral pixels. An n-th mesh point output value Bn, which is an output value of the n-th mesh point coordinates Cn in one piece of image data, is represented by the following formula (3).

$$Bn = ( rn , gn , bn ) \qquad ( 3 )$$

Here, rn is a red tone value contained in the output value of the mesh point coordinates corresponding to the n-th mesh point coordinates Cn. gn is a green tone value contained in the output value of the mesh point coordinates corresponding to the n-th mesh point coordinates Cn. bn is a blue tone value contained in the output value of the mesh point coordinates corresponding to the n-th mesh point coordinates Cn.

The data processing unit 35 generates n-th face feature point data Dn in which the n-th mesh point coordinates Cn in one piece of image data are associated with the n-th mesh point output value Bn. The n-th face feature point data Dn is an example of face feature point data. The face feature point data corresponds to an example of feature point data. The data processing unit 35 generates the n-th face feature point data Dn of the n-th mesh point coordinates Cn shown in the following formula (4). The n-th face feature point data Dn is generated for each of the N mesh point coordinates.

$$Dn = ( xn , yn , zn , rn , gn , bn ) \qquad ( 4 )$$

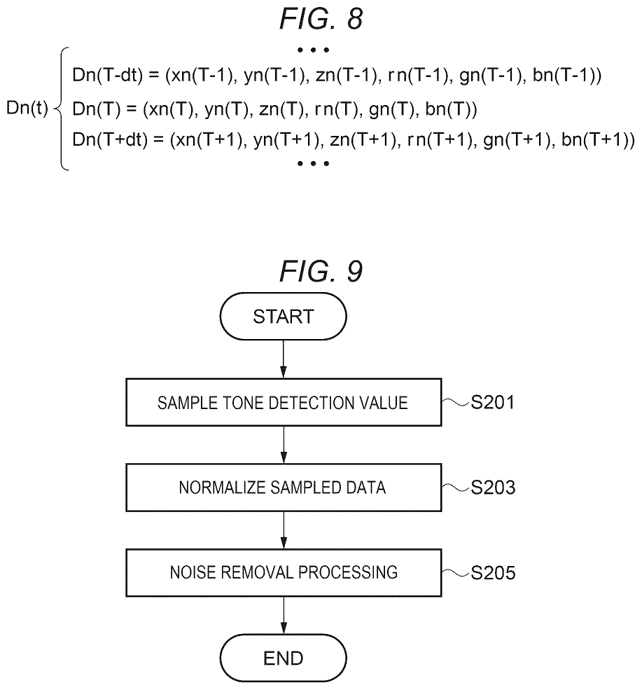

The data processing unit 35 acquires the n-th face feature point data Dn for each piece of image data. The data processing unit 35 generates n-th face feature point time series data Dn(t) by tracking the n-th face feature point data Dn for each piece of image data. The n-th face feature point time series data Dn(t) is an example of face feature point time series data of the n-th mesh point coordinates Cn. The face feature point time series data corresponds to an example of a feature point data group. The n-th face feature point time series data Dn(t) includes T time n-th face feature point data Dn(T), T-dt time n-th face feature point data Dn(T−dt), and T+dt time n-th face feature point data Dn(T+dt) acquired at a freely set time T at a frame rate of dt. The n-th face feature point time series data Dn(t) is shown in FIG. 8.

The data processing unit 35 acquires the n-th face feature point time series data Dn(t) shown in FIG. 8 by acquiring the n-th face feature point data Dn of the n-th mesh point coordinates Cn for each piece of image data. The n-th face feature point time series data Dn(t) is face feature point time series data of the n-th mesh point 131a tracked for each piece of image data.

After acquiring the output values of the mesh point coordinates, the measurement apparatus 10 determines whether the face feature point time series data of all mesh point coordinates are acquired in step S113. The data processing unit 35 determines whether all the n-th face feature point time series data Dn(t) in which n is 1 to N are acquired. When it is determined that the data processing unit 35 acquires all the n-th face feature point time series data Dn(t), the measurement apparatus 10 proceeds the processing to step S115 (step S113: YES). When it is determined that the data processing unit 35 does not acquire all the n-th face feature point time series data Dn(t), the measurement apparatus 10 returns the processing to step S109 (step S113: NO). The measurement apparatus 10 continues to acquire the n-th face feature point time series data Dn(t).

In step S115, the measurement apparatus 10 ends the acquisition of the face feature point time series data. The data processing unit 35 acquires the n-th face feature point time series data Dn(t) in which n is 1 to N. The data processing unit 35 acquires the n-th face feature point time series data Dn(t) including the n-th face feature point data Dn.

After acquiring the face feature point time series data, the measurement apparatus 10 selects a measurement point in step S117. The data processing unit 35 selects one or more mesh points 131a among the plurality of mesh points 131a as the measurement point. The measurement apparatus 10 acquires face feature point time series data corresponding to the measurement point as measurement point time series data. The measurement point time series data corresponds to an example of a feature point data group. The measurement point time series data includes a plurality of pieces of measurement point data which is face feature point data corresponding to the measurement point. The measurement point data corresponds to an example of measurement feature point data. The data processing unit 35 extracts measurement point data which is face feature point data of a measurement point by setting the measurement point. A method of setting the measurement point will be described later.

After setting the measurement point, the measurement apparatus 10 detects a pulse wave signal in step S119. The data processing unit 35 detects the pulse wave signal using the measurement point time series data or the measurement point data contained in the measurement point time series data. The measurement point time series data includes a plurality of pieces of measurement point data associated with the predetermined mesh point 131a. The data processing unit 35 generates a detection value using the measurement point data. For example, the data processing unit 35 detects the red tone detection value Dr, the green tone detection value Dg, and the blue tone detection value Db shown in FIG. 5 using the measurement point time series data.

FIG. 9 shows an example of an analysis procedure for detecting the pulse wave signal. FIG. 9 is a flowchart showing the example of the analysis procedure. The analysis procedure shown in FIG. 9 is executed by the data processing unit 35. In the analysis procedure shown in FIG. 9, the pulse wave signal is detected by using the red tone detection value Dr, the green tone detection value Dg, and the blue tone detection value Db.

In step S201, the data processing unit 35 samples tone detection values at a predetermined time interval. The time interval and a sampling frequency are set as appropriate. The time interval is preferably a time containing one or more pulse waves. The time interval is, for example, 3 seconds to 10 seconds. The sampling frequency is, for example, 10 Hz or more and 50 Hz or less. The data processing unit 35 acquires sampled data by performing the sampling. The sampled data contains the sampled red tone detection values Dr, green tone detection values Dg, and blue tone detection values Db.

After performing the sampling, the data processing unit 35 normalizes the sampled data in step S203. The data processing unit 35 normalizes the green tone detection value Dg contained in the sampled data.

The data processing unit 35 calculates a green average value Gmean, which is an average value of a plurality of the green tone detection values Dg, and a green standard deviation value Gstd, which is a standard deviation value of a plurality of the green tone detection values Dg. The data processing unit 35 normalizes each green tone detection value Dg using the following formula (5).

$$G n o r {m}_{m} = ( Gm - Gmean ) / Gstd \qquad ( 5 )$$

Here, m is any integer of 1 or more. Gm is the m-th green tone detection value Dg. Gnormₘ is a value obtained by normalizing the m-th green tone detection value Dg.

The data processing unit 35 normalizes a plurality of the red tone detection values Dr and a plurality of the blue tone detection values Db contained in the sampled data in a similar manner to the green tone detection value Dg. The data processing unit 35 calculates a red average value Rmean, which is an average value of the plurality of red tone detection values Dr, and a red standard deviation value Rstd, which is a standard deviation value of the plurality of red tone detection values Dr. The data processing unit 35 calculates a blue average value Bmean, which is an average value of a plurality of the blue tone detection values Db, and a blue standard deviation value Bstd, which is the standard deviation value of the plurality of blue tone detection values Db. The data processing unit 35 normalizes each of the red tone detection values Dr and each of the blue tone detection values Db by using the following formulas (6) and (7).

$$R n o r {m}_{m} = ( Rm - Rmean ) / Rstd \qquad ( 6 )$$

$${Bnorm}_{m} = ( Bm - Bmean ) / Bstd \qquad ( 7 )$$

Here, m is any integer of 1 or more. Rm is the m-th red tone detection value Dr. Rnormₘ is a value obtained by normalizing the m-th red tone detection value Dr. Bm is the m-th blue tone detection value Db. Bnormₘ is a value obtained by normalizing the m-th blue tone detection value Db.

After normalizing the sampled data, the data processing unit 35 executes noise removal processing in step S205. The data processing unit 35 executes the noise removal processing by using the normalized green tone detection values Dg, the normalized red tone detection values Dr, and the normalized blue tone detection values Db. The data processing unit 35 executes the noise removal processing using the following formula (8) to generate a noise-removed signal S.

$$Sm = {Gorm}_{m} + α {Bnorm}_{m} + β {Rnorm}_{m} \qquad ( 8 )$$

Here, m is any integer of 1 or more. Sm is an m-th noise-removed signal S. α is a first coefficient, and β is a second coefficient.

For example, α and β are each −0.5. When α and β are negative values, the data processing unit 35 detects the noise-removed signal S by subtracting the normalized red tone detection values Dr and the normalized blue tone detection values Db from the normalized green tone detection values Dg. At least one of α and β may be zero. When α=0 and β=−0.5, the data processing unit 35 detects the noise-removed signal S by calculating a difference between the green tone detection value Dg and the red tone detection value Dr. When α=−0.5 and β=0, the data processing unit 35 detects the noise-removed signal S by calculating a difference between the green tone detection value Dg and the blue tone detection value Db. The coefficients α and β are set as appropriate in accordance with a state of the noise removal.

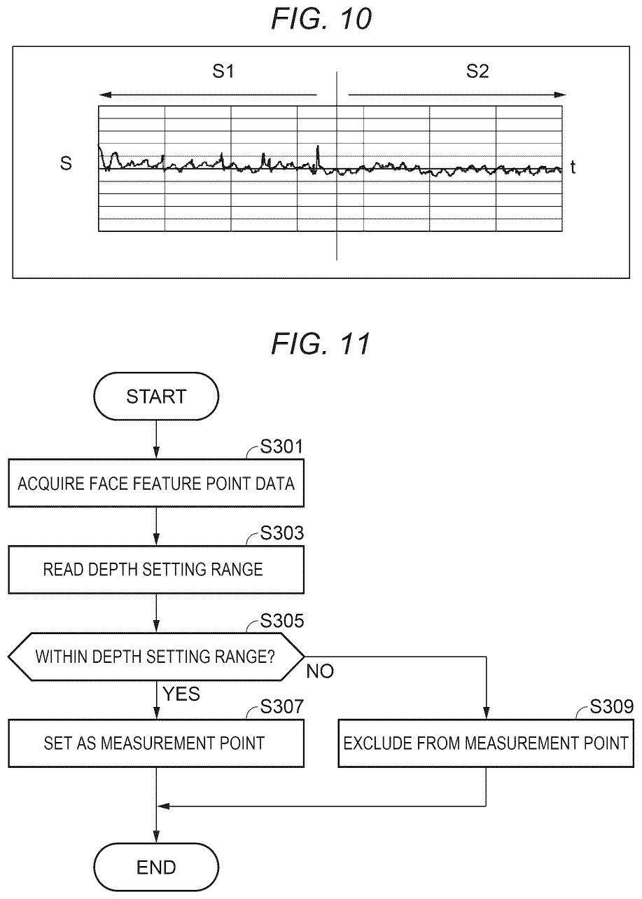

FIG. 10 shows an analysis result of the noise-removed signal S. FIG. 10 shows an analysis based on the red tone detection value Dr, the green tone detection value Dg, and the blue tone detection value Db shown in FIG. 5. FIG. 10 shows the noise-removed signal S when α=−0.5 and β=−0.5 are substituted into the formula (8). FIG. 10 shows the noise-removed signal S in the body motion section S1 and the rest section S2.

The noise-removed signal S corresponds to a pulse wave signal, as shown in FIG. 10. Noise components such as a body motion are removed from the noise-removed signal S. The data processing unit 35 detects the noise-removed signal S as a pulse wave signal. A signal waveform of the noise-removed signal S in the rest section S2 is detected more clearly than that of the green tone detection value Dg. The noise-removed signal S in the body motion section S1 is adjusted to a signal waveform corresponding to the pulse wave signal. By executing the noise removal processing, the data processing unit 35 can detect pulse wave signals in the body motion section S1 and the rest section S2.

The data processing unit 35 may calculate biometric information such as a pulse wave using the noise-removed signal S. The data processing unit 35 acquires the noise-removed signal S as a pulse wave signal. The data processing unit 35 calculates biometric information such as a pulse by calculating a cycle, an amplitude, and the like of the pulse wave signal. The data processing unit 35 transmits the biometric information including the pulse wave signal to the display control unit 37. The data processing unit 35 may store the biometric information and the like in the storage unit 41.

The data processing unit 35 acquires the pulse wave signal using one or any number of pieces of n-th face feature point time series data Dn(t) among N pieces of the n-th face feature point time series data Dn(t) in which n is 1 to N.

FIG. 11 shows an example of a control flow executed by the measurement apparatus 10. FIG. 11 shows an example of a method of setting a measurement point. FIG. 11 shows an example of the control flow executed in step S117 shown in FIG. 7. FIG. 11 shows a control flow for setting a measurement point using a depth component contained in mesh point coordinates.

In step S301, the measurement apparatus 10 acquires face feature point data. The data processing unit 35 acquires the face feature point data contained in the face feature point time series data. The data processing unit 35 acquires zn contained in the n-th face feature point data Dn. zn is a depth component of the n-th mesh point coordinates Cn.

After acquiring the face feature point data, the measurement apparatus 10 reads a depth setting range in step S303. The data processing unit 35 reads the depth setting range stored in the storage unit 41. The depth setting range is numerical range information that specifies a depth coordinate range of a depth component contained in mesh point coordinates of the mesh point 131a extracted as the measurement point. The depth setting range corresponds to an example of a depth range threshold. The depth setting range is stored in the storage unit 41 in advance. For example, the depth setting range is set by a depth coordinate minimum value zmin and a depth coordinate maximum value zmax. The depth coordinate minimum value zmin is a lower limit value of the depth coordinate range. The depth coordinate maximum value zmax is an upper limit value of the depth coordinate range.

After reading the depth setting range, the measurement apparatus 10 determines whether the depth component contained in the mesh point coordinates is within the depth setting range in step S305. The data processing unit 35 compares the depth component contained in the mesh point coordinates with the depth setting range. The data processing unit 35 determines whether the depth component contained in the mesh point coordinates of each mesh point 131a satisfies a relationship of the following formula (9).

$$z min < zn < z max \qquad ( 9 )$$

For example, an average value of a plurality of depth components is set to 0. A position closer to the imaging unit 11 than a position having the average value of the depth components is a negative value, and a position farther from the imaging unit 11 than the position having the average value of the depth components is a positive value. An edge portion of the face is smaller than 0. A depth component of a peripheral portion of a nose tip and a nose bridge of the face is larger than 0. Output values of parts such as the edge portion of the face, and the peripheral portion of the nose tip and the nose bridge tend to vary depending on an orientation of the face. Output values of parts such as the edge portion of the face, and the peripheral portion of the nose tip and the nose bridge are excluded from detection of a pulse wave signal according to the depth setting range. Detection accuracy of the pulse wave signal is improved by using the mesh point 131a at the mesh point coordinates within the depth setting range as the measurement point.

When zn contained in the n-th mesh point coordinates Cn satisfies the relationship of formula (9), the data processing unit 35 determines that the depth component is within the depth setting range. The measurement apparatus 10 proceeds the processing to step S307 (step S305: YES). When zn contained in the n-th mesh point coordinates Cn does not satisfy the relationship of formula (9), the data processing unit 35 determines that the depth component is not within the depth setting range. The measurement apparatus 10 proceeds the processing to step S309 (step S305: NO).

In step S305, the measurement apparatus 10 may normalize zn and then compare the normalized zn with the depth setting range. The depth setting range is set to a numerical range corresponding to the normalized zn. The data processing unit 35 normalizes zn using the following formula (10).

$$z n o r {m}_{n} = ( z / ( z max - z min ) ) n \qquad ( 10 )$$

Here, znormₙ represents the normalized zn. zmax is a maximum value of zn contained in the face feature point data. zmin is a minimum value of zn contained in the face feature point data.

The depth setting range corresponding to the normalized zn is, for example, a range of 25% above and below a median value. The depth setting range is appropriately set and stored in the storage unit 41. The data processing unit 35 determines whether the normalized zn is within the depth setting range.

In step S307, the measurement apparatus 10 sets the mesh point 131a whose depth component is within the depth setting range as the measurement point. The data processing unit 35 acquires the face feature point time series data of the mesh point 131a set as the measurement point as the measurement point time series data. The measurement point time series data includes a plurality of pieces of measurement point data. The data processing unit 35 detects a pulse wave signal in step S119 shown in FIG. 7 using the measurement point time series data including the plurality of pieces of measurement point data.

In step S309, the measurement apparatus 10 excludes the mesh point 131a whose depth component is not within the depth setting range from the measurement point. The data processing unit 35 does not use the face feature point time series data of the mesh point 131a excluded from the measurement point for detection of the pulse wave signal.

The data processing unit 35 compares zn contained in the n-th mesh point coordinates Cn in which n is 1 to N with the depth setting range in the control flow shown in FIG. 11. The data processing unit 35 sets one or more mesh points 131a as the measurement point by comparing zn with the depth setting range. The data processing unit 35 acquires the face feature point time series data of the measurement point as the measurement point time series data. The measurement point time series data includes a plurality of pieces of measurement point data.

The data processing unit 35 detects the pulse wave signal in step S119 shown in FIG. 7 using the measurement point time series data which is the face feature point time series data of one or more measurement points. When a plurality of measurement points are set, the data processing unit 35 acquires a plurality of pieces of face feature point data contained in the face feature point time series data of each measurement point. For example, the data processing unit 35 calculates an average mesh point output value Bave using a mesh point output value of a measurement point for each piece of image data. The average mesh point output value Bave is represented by the following formula (11).

$$Bave = ( rave , gave , bave ) \qquad ( 11 )$$

Here, rave is an average red tone value calculated based on an output value of each measurement point. gave is an average green tone value calculated based on an output value of each measurement point. bave is an average blue tone value calculated based on an output value of each measurement point.

The data processing unit 35 calculates average mesh point output value time series data Bave(t) using the average mesh point output value Bave. The average mesh point output value time series data Bave(t) is represented by the following formula (12).

$$Bave ( t ) = ( rave ( t ) , gave ( t ) , bave ( t ) ) \qquad ( 12 )$$

The data processing unit 35 acquires a pulse wave signal in step S119 shown in FIG. 7 using the average mesh point output value time series data Bave(t) calculated based on the face feature point time series data of each measurement point. The data processing unit 35 tracks the plurality of mesh points 131a and acquires the pulse wave signal using the face feature point data of the plurality of mesh points 131a, thereby improving measurement accuracy of the pulse wave signal.

The measurement apparatus 10 includes the imaging unit 11 that images the measurement operator M and generates moving image data including a plurality of pieces of image data, the image recognition processing unit 33 that identifies a face image contained in the image data and acquires mesh point coordinates indicating positions of the plurality of mesh points 131a contained in the face image, and the data processing unit 35 that generates a plurality of pieces of face feature point data in which the mesh point coordinates are associated with output values of pixels contained in the image data and detects a pulse wave signal of the measurement operator M based on the face feature point data. The mesh point coordinates include a width component, a height component, and a depth component. The data processing unit 35 extracts measurement point data from the plurality of pieces of face feature point data using the mesh point coordinates contained in the face feature point data, and detects the pulse wave signal using the measurement point data.

The measurement apparatus 10 detects the pulse wave signal using the measurement point data, thereby improving detection accuracy of the pulse wave signal.

The measurement apparatus 10 includes the storage unit 41 that stores the depth setting range for designating a depth coordinate range of a depth component. The data processing unit 35 preferably extracts the measurement point data by comparing a depth component contained in the mesh point coordinates with the depth setting range.

The data processing unit 35 extracts the measurement point data using the depth component contained in the mesh point coordinates and the depth setting range, so that the mesh point 131a in the face image region 121 where the detection accuracy of the pulse wave signal is reduced can be excluded from the measurement point. The detection accuracy of the pulse wave signal is improved.

The biometric analysis program PG causes the control unit 31, which is coupled to the imaging unit 11 that images the measurement operator M and generates a plurality of pieces of image data, to identify a face image contained in the image data, acquire mesh point coordinates indicating positions of the plurality of mesh points 131a contained in the face image, generate a plurality of pieces of face feature point data in which the mesh point coordinates are associated with output values of pixels contained in the image data, extract measurement point data from the plurality of pieces of face feature point data using the mesh point coordinates contained in the face feature point data, and detect a pulse wave signal using the measurement point data.

The biometric analysis program PG improves the detection accuracy of the pulse wave signal by detecting the pulse wave signal using the measurement point data.

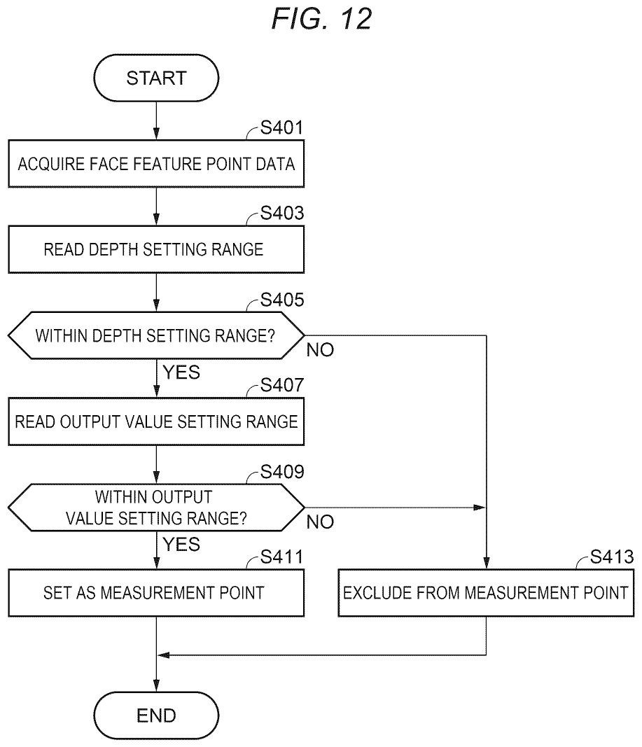

FIG. 12 shows an example of a control flow executed by the measurement apparatus 10. FIG. 12 shows an example of a method of setting a measurement point. FIG. 12 shows an example of the control flow executed in step S117 shown in FIG. 7. FIG. 12 shows a control flow for setting a measurement point using a depth component contained in mesh point coordinates and an output value.

In step S401, the measurement apparatus 10 acquires face feature point data. The data processing unit 35 acquires the face feature point data contained in the face feature point time series data. The data processing unit 35 acquires zn contained in the n-th face feature point data Dn. zn is a depth component of the n-th mesh point coordinates Cn.

After acquiring the face feature point data, the measurement apparatus 10 reads a depth setting range in step S403. The data processing unit 35 reads the depth setting range stored in the storage unit 41.

After reading the depth setting range, the measurement apparatus 10 determines whether the depth component of the mesh point coordinates is within the depth setting range in step S405. The data processing unit 35 compares the depth component of the mesh point coordinates with the depth setting range. The data processing unit 35 determines whether the depth component contained in the mesh point coordinates of each mesh point 131a satisfies the relationship of formula (9).

When zn contained in the n-th mesh point coordinates Cn satisfies the relationship of formula (9), the data processing unit 35 determines that the depth component is within the depth setting range. The measurement apparatus 10 proceeds the processing to step S407 (step S405: YES). When zn contained in the n-th mesh point coordinates Cn does not satisfy the relationship of formula (9), the data processing unit 35 determines that the depth component is not within the depth setting range. The measurement apparatus 10 proceeds the processing to step S413 (step S405: NO).

In step S407, the measurement apparatus 10 reads an output value setting range. The data processing unit 35 reads the output value setting range from the storage unit 41. The output value setting range corresponds to an example of a light amount threshold. The output value setting range is stored in the storage unit 41 in advance. The output value setting range is information for designating an output value range of an output value associated with the mesh point coordinates. The output value range corresponds to an example of a light amount range. The output value setting range includes information for specifying a lower limit value of at least one of the red tone value, the green tone value, and the blue tone value contained in the face feature point data. The output value setting range is compared with the output value associated with the mesh point coordinates.

After reading the output value setting range, the measurement apparatus 10 determines whether the output value is within the output value setting range in step S409. In step S409, the data processing unit 35 compares the output value corresponding to the mesh point coordinates whose depth component is within the depth setting range with the output value setting range. The data processing unit 35 compares at least one of the red tone value, the green tone value, and the blue tone value contained in the face feature point data of each mesh point 131a with the output value setting range.

When the data processing unit 35 determines that the output value is within the output value setting range, the measurement apparatus 10 proceeds the processing to step S411 (step S409: YES). When the data processing unit 35 determines that the output value is not within the output value setting range, the measurement apparatus 10 proceeds the processing to step S413 (step S409: NO).

In step S411, the measurement apparatus 10 sets, as a measurement point, the mesh point 131a of the face feature point data whose depth component is within the depth setting range and whose output value is within the output value setting range. The data processing unit 35 acquires face feature point time series data of the mesh point 131a set as the measurement point.

In step S413, the measurement apparatus 10 excludes the mesh point 131a a of face feature point data whose depth component is not within the depth setting range and the mesh point 131a of face feature point data whose output value is not within the output value setting range from the measurement point. The data processing unit 35 does not use the face feature point time series data of the mesh point 131a excluded from the measurement point for detection of the pulse wave signal.

The data processing unit 35 executes the control flow shown in FIG. 12 on the n-th face feature point data Dn in which n is 1 to N, and sets one or more mesh points 131a as a measurement point. The data processing unit 35 extracts face feature point time series data corresponding to one or more measurement points as measurement point time series data. One or more pieces of the measurement point time series data include a plurality of pieces of measurement point data. In step S119 shown in FIG. 7, a pulse wave signal is detected using the measurement point time series data including the measurement point data.

The storage unit 41 stores the output value setting range for specifying an output value range of output values. The data processing unit 35 preferably extracts the measurement point data by comparing an output value contained in the face feature point data with the output value setting range.

The measurement apparatus 10 can exclude an output value of the mesh point 131a at a position where a shadow is generated depending on a position of a light source or the like. The measurement accuracy of the pulse wave signal is improved.

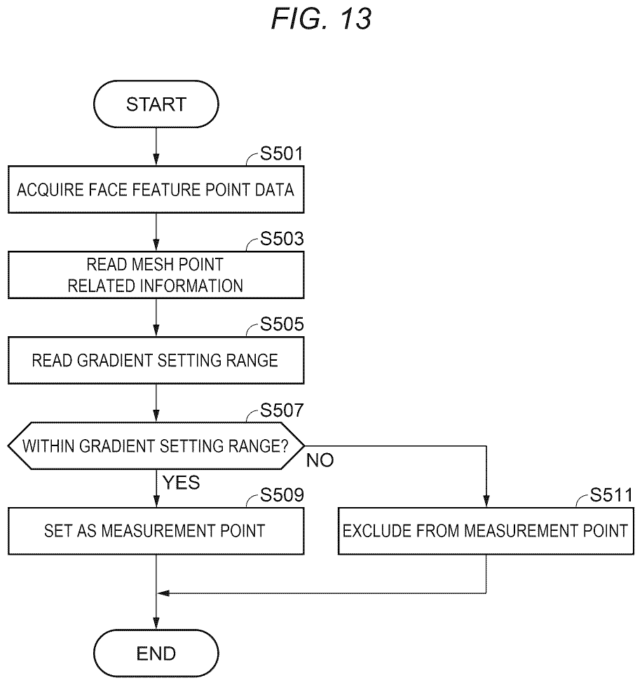

FIG. 13 shows an example of a control flow executed by the measurement apparatus 10. FIG. 13 shows an example of a method of setting a measurement point. FIG. 13 shows an example of the control flow executed in step S117 shown in FIG. 7. FIG. 13 shows a control flow for setting a measurement point using the mesh point related information MT and a depth component contained in mesh point coordinates.

In step S501, the measurement apparatus 10 acquires face feature point data. The data processing unit 35 acquires the face feature point data contained in the face feature point time series data. The data processing unit 35 acquires the n-th mesh point coordinates Cn contained in the n-th face feature point data Dn.

In step S503, the measurement apparatus 10 reads the mesh point related information MT from the storage unit 41. For example, the data processing unit 35 reads the mesh point related information MT shown in FIG. 6.

In step S505, the measurement apparatus 10 reads a gradient setting range. The gradient setting range is numerical range information for specifying an inclination range of a depth component contained in mesh point coordinates of the adjacent mesh points 131a. The gradient setting range is stored in the storage unit 41 in advance. For example, the gradient setting range is set by a set gradient minimum value gmin and a set gradient maximum value gmax. The set gradient minimum value gmin is a lower limit value of the gradient setting range. The set gradient maximum value gmax is an upper limit value of the gradient setting range.

After reading the gradient setting range, in step S507, the measurement apparatus 10 determines whether a gradient of two adjacent mesh points 131a is within the gradient setting range. The gradient of two adjacent mesh points 131a is represented by the following formula (13).

$$g Δ n = ( zn - z ( n + 1 ) ) / d Δ n \qquad ( 13 )$$

Here, gΔn is a gradient between the mesh point 131a at the n-th mesh point coordinates Cn and the mesh point 131a at the adjacent (n+1)-th mesh point coordinates C(n+1). z(n+1) is a depth component contained in the (n+1)-th mesh point coordinates C(n+1). dΔn is a distance between the mesh point 131a at the n-th mesh point coordinates Cn and the mesh point 131a at the (n+1)-th mesh point coordinates C(n+1).

The data processing unit 35 compares a gradient of the two adjacent mesh points 131a with the gradient setting range. The data processing unit 35 determines whether the gradient of the two adjacent mesh points 131a satisfies a relationship of the following formula (14).

$$g min < g Δ n < g max \qquad ( 14 )$$

In the edge portion of the face and the peripheral portion of the nose tip and the nose bridge, the gradient of the two adjacent mesh points 131a has a value outside the gradient setting range. By setting the mesh point 131a within the gradient setting range as the measurement point, a possibility that an output value is detected within a predetermined range is improved. The detection accuracy of the pulse wave signal is prevented from being lowered.

When the gradient between one mesh point 131a and the mesh point 131a adjacent to the one mesh point 131a satisfies the relationship of the formula (14), the data processing unit 35 determines that the one mesh point 131a is located at mesh point coordinates within the gradient setting range with respect to the adjacent mesh point 131a. The data processing unit 35 determines that the one mesh point 131a can be set as a measurement point. The measurement apparatus 10 proceeds the processing to step S509 (step S507: YES). When the gradient between the one mesh point 131a and the adjacent mesh point 131a does not satisfy the relationship of formula (14), the data processing unit 35 determines that the one mesh point 131a is not within the gradient setting range with respect to the adjacent mesh point 131a. The data processing unit 35 determines not to extract the one mesh point 131a as a measurement point. The measurement apparatus 10 proceeds the processing to step S511 (step S507: NO).

In step S509, the measurement apparatus 10 sets the one mesh point 131a as a measurement point. The data processing unit 35 acquires the face feature point time series data of the mesh point 131a, which is set as the measurement point, as the measurement point time series data. The measurement point time series data includes a plurality of pieces of measurement point data. The data processing unit 35 detects a pulse wave signal in step S119 shown in FIG. 7 using the measurement point time series data including the measurement point data.

In step S511, the measurement apparatus 10 excludes the one mesh point 131a from a measurement point. The data processing unit 35 does not use the face feature point time series data of the mesh point 131a excluded from the measurement point for detection of the pulse wave signal.

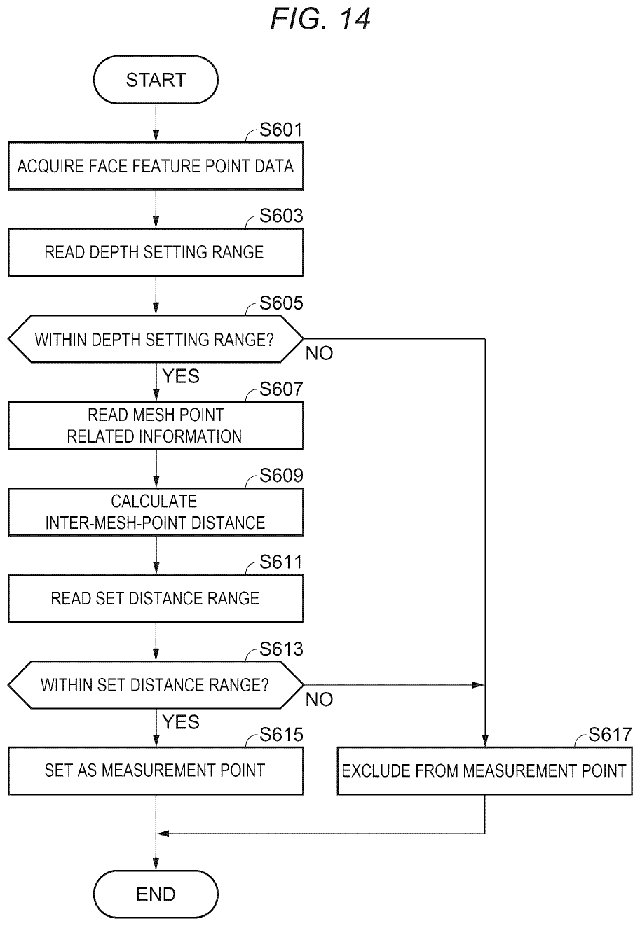

FIG. 14 shows an example of a control flow executed by the measurement apparatus 10. FIG. 14 shows an example of a method of setting a measurement point. FIG. 14 shows an example of the control flow executed in step S117 shown in FIG. 7. FIG. 14 shows a control flow for setting a measurement point using a width component, a height component, and a depth component contained in mesh point coordinates.

In step S601, the measurement apparatus 10 acquires face feature point data. The data processing unit 35 acquires the face feature point data contained in the face feature point time series data. The data processing unit 35 acquires zn contained in the n-th face feature point data Dn.

After acquiring the face feature point data, the measurement apparatus 10 reads a depth setting range in step S603. The data processing unit 35 reads the depth setting range stored in the storage unit 41.

After reading the depth setting range, the measurement apparatus 10 determines whether the depth component of the mesh point coordinates is within the depth setting range in step S605. The data processing unit 35 compares the depth component of the mesh point coordinates with the depth setting range. The data processing unit 35 determines whether the depth component contained in the mesh point coordinates of each mesh point 131a satisfies the relationship of formula (9).

When zn contained in the n-th mesh point coordinates Cn satisfies the relationship of formula (9), the data processing unit 35 determines that the depth component is within the depth setting range. The measurement apparatus 10 proceeds the processing to step S607 (step S605: YES). When zn contained in the n-th mesh point coordinates Cn does not satisfy the relationship of formula (9), the data processing unit 35 determines that the depth component is not within the depth setting range. The measurement apparatus 10 proceeds the processing to step S617 (step S605: NO).

In step S607, the measurement apparatus 10 reads the mesh point related information MT from the storage unit 41. For example, the data processing unit 35 reads the mesh point related information MT shown in FIG. 6.

After reading the mesh point related information MT, the measurement apparatus 10 calculates an inter-mesh-point distance in step S609. The data processing unit 35 uses the mesh point related information MT to determine the mesh point 131a whose depth component is within the depth setting range and the adjacent mesh point 131a. The data processing unit 35 calculates the inter-mesh-point distance by using mesh point coordinates of the mesh point 131a whose depth component is within the depth setting range and mesh point coordinates of the adjacent mesh point 131a. The inter-mesh-point distance is a distance between the mesh point 131a whose depth component is within the depth setting range and the adjacent mesh point 131a. Here, the mesh point 131a whose depth component is within the depth setting range is represented as an i-th mesh point as an example. The adjacent mesh point 131a adjacent to the i-th mesh point is represented as a j-th mesh point. i-th mesh point coordinates Ci of the i-th mesh point and j-th mesh point coordinates Cj of the j-th mesh point are represented by the following formulas (15) and (16), respectively.

$$Ci = ( xi , yi , zi ) \qquad ( 15 )$$

$$Cj = ( xj , yj , zj ) \qquad ( 16 )$$

The data processing unit 35 calculates an ij inter-mesh-point distance dij using the i-th mesh point coordinates Ci and the j-th mesh point coordinates Cj. The ij inter-mesh-point distance dij is an example of an inter-mesh-point distance. The ij inter-mesh-point distance dij is represented by a formula (17).

$${d}_{ij} = \sqrt{{( {x}_{i} - {x}_{j} )}^{2} + {( {y}_{i} - {y}_{j} )}^{2}} \qquad ( 17 )$$

i and j are two different points of 1 to N.

In step S611, the measurement apparatus 10 reads a set distance range. The data processing unit 35 reads the set distance range from the storage unit 41. The set distance range corresponds to an example of a plane coordinate threshold. The set distance range is stored in the storage unit 41 in advance. The set distance range is information for designating a plane range related to a width component and a height component of mesh point coordinates. The plane range corresponds to an example of a plane coordinate range. The set distance range is compared with the inter-mesh-point distance calculated using the width component and the height component contained in the mesh point coordinates.

After reading the set distance range, the measurement apparatus 10 determines whether the inter-mesh-point distance is within the set distance range in step S613. When the inter-mesh-point distance is within the set distance range, two adjacent mesh points 131a are located at positions visible from the imaging unit 11 in a plan view. The data processing unit 35 determines that the two adjacent mesh points 131a are not moved to invisible positions due to an orientation of the face or the like. When the ij inter-mesh-point distance dij is within the set distance range, the data processing unit 35 determines that the i-th mesh point can be set as the measurement point. The measurement apparatus 10 proceeds the processing to step S615 (step S613: YES). When the inter-mesh-point distance is not within the set distance range, one of the two adjacent mesh points 131a is not at a position visible from the imaging unit 11 in a plan view. The data processing unit 35 determines that one of the two adjacent mesh points 131a is moved to an invisible position due to an orientation of the face or the like. When the ij inter-mesh-point distance dij is not within the set distance range, the data processing unit 35 determines that the i-th mesh point cannot be set as a measurement point. The measurement apparatus 10 proceeds the processing to step S617 (step S613: NO).

In step S615, the measurement apparatus 10 sets the i-th mesh point as a measurement point. The data processing unit 35 acquires the face feature point time series data of the i-th mesh point, which is set as the measurement point, as the measurement point time series data. The data processing unit 35 detects a pulse wave signal in step S119 shown in FIG. 7 using the measurement point time series data including the plurality of pieces of measurement point data.

In step S617, the measurement apparatus 10 excludes the i-th mesh point and the mesh point 131a whose depth component is not within the depth setting range from a measurement point. The data processing unit 35 does not use the face feature point time series data of the mesh point 131a whose depth component is not within the depth setting range and the face feature point time series data of the i-th mesh point for detection of a pulse wave signal.

In the control flow shown in FIG. 14, after it is determined whether the depth component is within the depth setting range, it is determined whether the inter-mesh-point distance is within the set distance range, but the present disclosure is not limited thereto. The measurement apparatus 10 may determine whether the depth component is within the depth setting range and determine whether the inter-mesh-point distance is within the set distance range at the same timing. The measurement apparatus 10 may determine whether the depth component is within the depth setting range after determining whether the inter-mesh-point distance is within the set distance range.

The storage unit 41 stores a set distance range for designating a plane range related to a width component and a height component. The data processing unit 35 preferably extracts the measurement point data using the set distance range.

The measurement apparatus 10 can exclude, from a measurement point, the mesh point 131a that is not located at a position visible from the imaging unit 11 in a plan view. The detection accuracy of the pulse wave signal is improved.

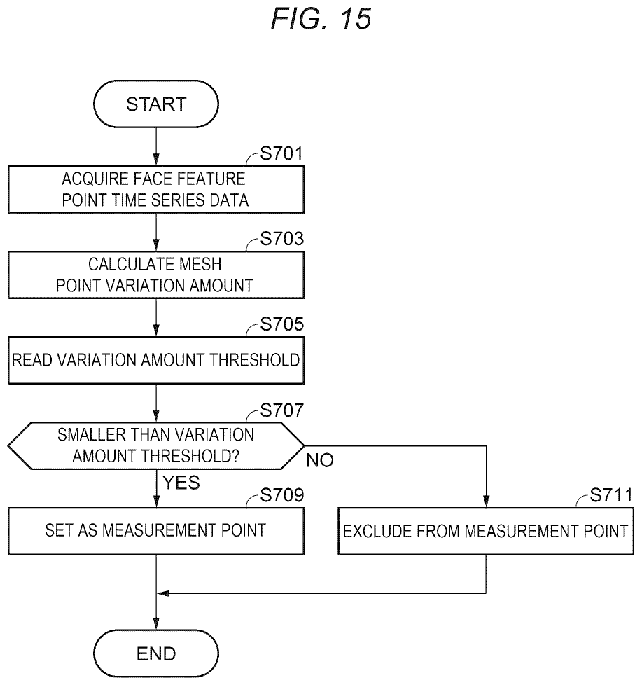

FIG. 15 shows an example of a control flow executed by the measurement apparatus 10. FIG. 15 shows an example of a method of setting a measurement point. FIG. 15 shows an example of the control flow executed in step S117 shown in FIG. 7. FIG. 15 shows a control flow for extracting measurement point data using face feature point time series data.

In step S701, the measurement apparatus 10 acquires face feature point time series data. The data processing unit 35 acquires face feature point time series data including face feature point data. The data processing unit 35 acquires the n-th face feature point time series data Dn(t).

After acquiring the face feature point time series data, the measurement apparatus 10 calculates a mesh point variation amount in step S703. The mesh point variation amount corresponds to an example of a variation amount. The mesh point variation amount indicates a variation within a measurement time of mesh point coordinates. The mesh point variation amount is a body motion index indicating the magnitude of a body motion of the measurement operator M. When the body motion increases, measurement accuracy of a pulse wave signal is lowered. The mesh point variation amount is represented by a formula (18) as an example. RMS in the formula (18) represents a root mean square.

$${RSM ( x , y , z )}_{n} = {( \sqrt{{\sum }_{t = T}^{T + τ} \frac{{( x - {x}_{ave} )}^{2}}{τ}} , \sqrt{{\sum }_{t = T}^{T + τ} \frac{{( y - {y}_{ave} )}^{2}}{τ}} , \sqrt{{\sum }_{t = T}^{T + τ} \frac{{( z - {z}_{ave} )}^{2}}{τ}} )}_{n} \qquad ( 18 )$$

n=1 to N

Here, xave is an average value of width components contained in the n-th face feature point data Dn. yave is an average value of height components contained in the n-th face feature point data Dn. zave is an average value of depth components contained in the n-th face feature point data Dn.

The mesh point variation amount may be calculated using a root mean square or a statistical value different from the root mean square. The mesh point variation amount may be calculated using a variance, a standard deviation, or a variation coefficient. A method of calculating the mesh point variation amount is appropriately set.

In step S705, the measurement apparatus 10 reads a variation t threshold from the storage unit 41. The variation amount threshold is stored in the storage unit 41 in advance. The variation amount threshold is compared with a mesh point variation amount. The variation amount threshold is set to a predetermined value in advance. When the mesh point variation amount is smaller than the variation amount threshold, it indicates that a body motion of the measurement operator M is in a range in which a pulse wave signal can be measured. When the mesh point variation amount is larger than the variation amount threshold, it indicates that a body motion of the measurement operator M is in a range in which a pulse wave signal is less likely to be detected.

After reading the variation amount threshold, the measurement apparatus 10 determines whether the mesh point variation amount is smaller than the variation amount threshold in step S707. The data processing unit 35 compares the mesh point variation amount with the variation amount threshold. When the data processing unit 35 determines that the mesh point variation amount is smaller than the variation amount threshold, the measurement apparatus 10 proceeds the processing to step S709 (step S707: YES). When the data processing unit 35 determines that the mesh point variation amount is larger than the variation amount threshold, the measurement apparatus 10 proceeds the processing to step S711 (step S707: NO).

In step S709, the measurement apparatus 10 sets the mesh point 131a whose mesh point variation amount is smaller than the variation amount threshold as a measurement point. The data processing unit 35 acquires the face feature point time series data of the mesh point 131a, which is set as the measurement point, as the measurement point time series data. The data processing unit 35 detects a pulse wave signal in step S119 shown in FIG. 7 using the measurement point time series data including the plurality of pieces of measurement point data.

In step S711, the measurement apparatus 10 excludes the mesh point 131a whose mesh point variation amount is larger than the variation amount threshold from the measurement point. The data processing unit 35 does not use the face feature point time series data of the mesh point 131a excluded from the measurement point for detection of the pulse wave signal. The detection accuracy of a pulse wave signal is improved by excluding the face feature point time series data of the mesh point 131a whose mesh point variation amount is larger than the variation amount threshold.

The data processing unit 35 generates the face feature point time series data by tracking the mesh points 131a contained in a face image in time series.

The data processing unit 35 improves the detection accuracy of the pulse wave signal by using the face feature point time series data of the tracked mesh points 131a.

It is preferable that the data processing unit 35 calculates the mesh point variation amount of mesh point coordinates in a plurality of pieces of face feature point data contained in the face feature point time series data, and extracts the measurement point data based on the mesh point variation amount.

The measurement apparatus 10 can improve the detection accuracy of the pulse wave signal by excluding the mesh point 131a whose mesh point variation amount is larger than the variation amount threshold.

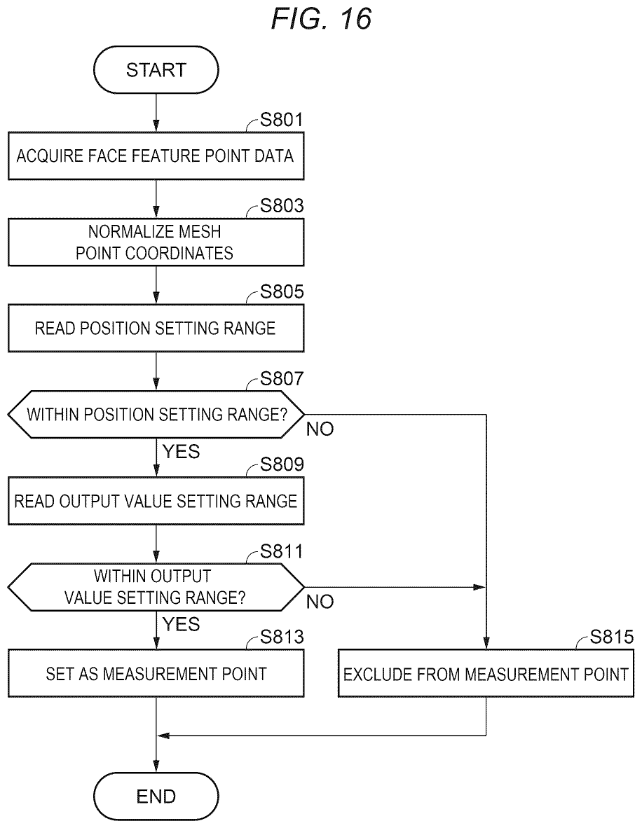

FIG. 16 shows an example of a control flow executed by the measurement apparatus 10. FIG. 16 shows an example of a method of setting a measurement point. FIG. 16 shows an example of the control flow executed in step S117 shown in FIG. 7. FIG. 16 shows a control flow for extracting measurement point data using mesh point coordinates contained in face feature point data.

In step S801, the measurement apparatus 10 acquires face feature point data. The data processing unit 35 acquires the face feature point data contained in the face feature point time series data. The data processing unit 35 acquires the n-th mesh point coordinates Cn contained in the n-th face feature point data Dn.

After acquiring the face feature point data, the measurement apparatus 10 normalizes the mesh point coordinates in step S803. The data processing unit 35 normalizes the n-th mesh point coordinates Cn in which n is 1 to N. For example, a calculation formula for normalizing the n-th mesh point coordinates Cn is represented by a formula (19). The normalized n-th mesh point coordinates Cn are shown in the n-th mesh point face feature point data Dn of the formula (19).

$${Dnorm}_{n} = {( {( \frac{x}{{x}_{max} - {x}_{min}} )}_{n} , {( \frac{y}{{y}_{max} - {y}_{min}} )}_{n} , \{ \frac{z}{{z}_{max} - {z}_{min}} )}_{n} , {r}_{n} , {g}_{n} , {b}_{n} ) \qquad ( 19 )$$

n=1 to N

Dnormₙ indicates the normalized n-th mesh point face feature point data Dn. Here, xmax is a maximum value of xn in which n is 1 to N. xmin is a minimum value of xn in which n is 1 to N. ymax is a maximum value of yn in which n is 1 to N. ymin is a minimum value of yn in which n is 1 to N. zmax is a maximum value of zn in which n is 1 to N. zmin is a minimum value of zn in which n is 1 to N.

After normalizing the mesh point coordinates, the measurement apparatus 10 reads a position setting range in step S805. The data processing unit 35 reads the position setting range from the storage unit 41. The position setting range is stored in the storage unit 41 in advance. The position setting range is information for designating a setting range related to a width component, a height component, and a depth component of the normalized mesh point coordinates. For example, the position setting range is a range of 25% above and below a median value of each of the normalized width component, height component, and depth component. The position setting range is a setting range for setting a value of at least one of a width component, a height component, and a depth component. By using the position setting range, the data processing unit 35 can exclude, from a measurement point, the mesh points 131a in a region where a pulse wave signal is less likely to be detected, such as a peripheral portion of the face, the periphery of the nose, and the orbit.

After reading the position setting range, the measurement apparatus 10 determines whether the normalized mesh point coordinates are within the position setting range in step S807. The measurement apparatus 10 compares the normalized mesh point coordinates with the position setting range. When the data processing unit 35 determines that the normalized mesh point coordinates are within the position setting range, the measurement apparatus 10 proceeds the processing to step S809 (step S807: YES). When the data processing unit 35 determines that the normalized mesh point coordinates are not within the position setting range, the measurement apparatus 10 proceeds the processing to step S815 (step S807: NO).

In step S809, the measurement apparatus 10 reads an output value setting range. The data processing unit 35 reads the output value setting range from the storage unit 41. The output value setting range is stored in the storage unit 41 in advance. The output value setting range is information for designating an output value range of an output value associated with the mesh point coordinates. The output value setting range includes information for specifying a lower limit value of at least one of the red tone value, the green tone value, and the blue tone value contained in the face feature point data. The output value setting range is compared with the output value associated with the mesh point coordinates.

After reading the output value setting range, the measurement apparatus 10 determines whether the output value is within the output value setting range in step S811. In step S811, the data processing unit 35 compares the output value of the mesh point 131a whose mesh point coordinates are within the position setting range with the output value setting range. The data processing unit 35 compares the output value setting range with the red tone value, the green tone value, and the blue tone value contained in the face feature point data of the mesh point 131a whose mesh point coordinates are within the position setting range.

When the data processing unit 35 determines that the output value is within the output value setting range, the measurement apparatus 10 proceeds the processing to step S813 (step S811: YES). When the data processing unit 35 determines that the output value is not within the output value setting range, the measurement apparatus 10 proceeds the processing to step S815 (step S811: NO).

In step S813, the measurement apparatus 10 sets, as a measurement point, the mesh point 131a whose mesh point coordinates are within the position setting range and whose output value is within the output value setting range. The data processing unit 35 acquires the face feature point time series data of the mesh point 131a, which is set as the measurement point, as the measurement point time series data. The data processing unit 35 detects a pulse wave signal in step S119 shown in FIG. 7 using the measurement point time series data including a plurality of pieces of measurement point data.

In step S815, the measurement apparatus 10 excludes the mesh point 131a whose mesh point coordinates are not within the position setting range and the mesh point 131a whose output value is not within the output value setting range from the measurement point. The data processing unit 35 does not use the face feature point time series data of the mesh point 131a excluded from the measurement point for detection of the pulse wave signal.

In the control flow shown in FIG. 16, the measurement point is set by determining whether the output value is within the output value setting range, but the present disclosure is not limited thereto. The control flow for setting a measurement point may not include step S809 and step S811. The measurement apparatus 10 may determine whether the mesh point coordinates are within the position setting range and set a measurement point based on a determination result.

The control flows shown in FIGS. 12, 13, 14, 15, and 16 can be changed as appropriate. For example, the measurement apparatus 10 may execute the processing of step S301 after executing the processing of step S303 in the control flow shown in FIG. 11. An execution order of the steps is appropriately set.

What is claimed is:

1. A biometric information acquisition apparatus comprising:
an imaging unit configured to image a living body and generate imaging data including image data;
a face recognition unit configured to identify a face image contained in the image data and acquire three-dimensional coordinate data indicating positions of a plurality of feature points contained in the face image; and
a detection unit configured to generate a plurality of pieces of feature point data in which the three-dimensional coordinate data is associated with a light amount value of a pixel contained in the image data, extract measurement feature point data for detecting a pulse wave signal of the living body from the plurality of pieces of feature point data by using the three-dimensional coordinate data, and detect the pulse wave signal using the measurement feature point data.

2. The biometric information acquisition apparatus according to claim 1, further comprising:
a storage unit configured to store a depth range threshold for designating a depth coordinate range for a depth component contained in the three-dimensional coordinate data, wherein
the detection unit extracts the measurement feature point data by comparing the depth component contained in the three-dimensional coordinate data with the depth range threshold.

3. The biometric information acquisition apparatus according to claim 2, wherein
the storage unit stores a plane coordinate threshold for designating a plane coordinate range for a width component of the three-dimensional coordinate data and a height component of the three-dimensional coordinate data, and
the detection unit extracts the measurement feature point data using the plane coordinate threshold.

4. The biometric information acquisition apparatus according to claim 2, wherein
the storage unit stores a light amount threshold for designating a light amount range of the light amount value, and
the detection unit extracts the measurement feature point data by comparing the light amount value contained in each of the plurality of pieces of feature point data with the light amount threshold.

5. The biometric information acquisition apparatus according to claim 1, wherein
the detection unit generates a feature point data group by tracking the feature points contained in the face image in time series.

6. The biometric information acquisition apparatus according to claim 5, wherein
the detection unit calculates a variation amount of the three-dimensional coordinate data for each of the plurality of pieces of feature point data, and extracts the measurement feature point data from the plurality of pieces of feature point data based on the variation amount.

7. A non-transitory computer-readable storage medium storing a biometric information acquisition program for causing a computer, which is coupled to an imaging unit that images a living body and generates image data, to execute the following processing of:
identifying a face image contained in the image data;
acquiring three-dimensional coordinate data indicating positions of a plurality of feature points contained in the face image;
generating a plurality of pieces of feature point data in which the three-dimensional coordinate data is associated with a light amount value of a pixel contained in the image data;
extracting measurement feature point data for detecting a pulse wave signal of the living body from the plurality of pieces of feature point data by using the three-dimensional coordinate data; and
detecting the pulse wave signal using the measurement feature point data.
